(19) United States
(12) Patent Application Publication
(10) Pub. No.: US 2025/0205738 A1
(43) Pub. Date: Jun. 26, 2025

(54) A HIGH FREQUENCY, HIGH RESOLUTION 2D PHASED ARRAY ULTRASONIC TRANSDUCER

(71) Applicants: NEWSOUTH INNOVATIONS PTY LIMITED, Sydney, New South Wales (AU); THE HONG KONG POLYTECHNIC UNIVERSITY, Hung Hom Kowloon, HK (CN)

(72) Inventors: Danyang WANG, Sydney, New South Wales (AU); Weichang WU, Sydney, New South Wales (AU); Jiyang DAI, Hung Hom Kowloon (CN); Sean Suixiang LI, Sydney, New South Wales (AU)

(21) Appl. No.: 18/852,107

(22) PCT Filed: Mar. 30, 2023
(86) PCT No.: PCT/AU2023/050241

§ 371 Date: Sep. 27, 2024

(30) Foreign Application Priority Data
Mar. 30, 2022 (AU) 2022900809

(51) Int. Cl. B06B 1/06; G10K 11/30; H10N 30/063; H10N 30/088; H10N 30/20; H10N 30/80; H10N 30/87; H10N 39/00

(52) U.S. Cl. CPC B06B 1/0629; G10K 11/30; H10N 30/063; H10N 30/088; H10N 30/2042; H10N 30/802; H10N 30/874; H10N 39/00

(57) ABSTRACT

A 2D phased array ultrasound device possessing ultrahigh spatial resolutions (<90 μm) and high centre frequency (25 MHz) including: a 2D array of piezo crystal elements formed from the kerfing of a single crystal; a series of conductive electrodes formed on opposed sides of the piezo crystal elements; a series of front matching layers; and a backing unit comprising backing filler material and a series of flexible circuit layers sandwich together to interconnect the back surface electrodes of the piezo crystal elements.

### RELATED APPLICATION

The present disclosure claims benefit of priority to Australian Provisional Patent Application Number: 2022900809 filed 30 Mar. 2022, entitled: “A high frequency, high resolution 2D phased array ultrasonic transducer”, the contents of which are incorporated herein by reference. In jurisdictions where incorporation by reference is not permitted, the applicant reserves the right to add any or the whole of the contents of said Application 2022900809 as an Appendix hereto, forming part of the specification.

### FIELD OF THE INVENTION

The present invention provides for systems and methods for providing an improved form of high-frequency 2D phased array ultrasound transducer.

### BACKGROUND OF THE INVENTION

Any discussion of the background art throughout the specification should in no way be considered as an admission that such art is widely known or forms part of common general knowledge in the field.

As a non-invasive and radiation-free means, ultrasound has been widely used as a medical diagnostic tool, owing to their strong ability of imaging the structure of an object which is hidden from view. Array ultrasonic transducers employ a number of individual active elements organized as either a one dimensional (1D or linear) array or a two-dimensional array, which can be properly excited, allowing the control and shaping of the ultrasonic beam in a specific manner. The medical array transducers are normally required to work at a frequency ranging from one to several tens MHz in order to obtain high-resolution images.

Both 1D linear and 2D array ultrasonic transducers are capable of producing 3D images. In ID linear array transducers, multiple cross-sectional 2D images are acquired through manually wobbling the transducer. Thus, 3D images are digitally rebuilt by a number of individual 2D images. Therefore, the rebuilt 3D images cannot reflect the real-time behaviours and functions of the human organs. In marked contrast, 2D array transducers can generate 3D images directly by arranging the piezoelectric elements in the form of a 2D array without requiring movement of the transducer. Linear arrays have a low scanning rate given that 3D images are created through mechanical sequential acquisition of 2D images in real-time, though they are easy to fabricate. However, 2D array transducers are highly desired for true real-time 3D images. Nevertheless, manufacturing of high resolution 2D array transducers remains very challenging. In particular, in the process of element fabrication and interconnection between transducers and imaging system.

The fabrication of high-frequency single-crystal 2D phased array transducers remains highly challenging and expensive primarily due to two reasons. In the case that a large number of elements (from 256-4096 or more) are needed to meet the requirements of higher resolution and larger scanning area, wiring of the elements becomes extremely difficult. Each individual element must be properly wired to allow them being independently controlled by corresponding electronic channels. It is extremely difficult to wire thousands of the elements in a one-by-one manner given the limited cross-sectional areas of array-ranged elements. On the other hand, most commercial 2D phased array ultrasonic transducers employ conventional PZT ceramics as the active layer and operate at low frequencies. Thus, the other key issue in the fabrication of high-frequency 2D array transducer is associated with the fragility of piezoelectric single crystals, which may result in the damage and partial de-poling of the elements in the machining process, e.g. cutting, polishing, lapping and wire-soldering, considering the element pitch size is rather small for high frequency application (>20 MHz).

The 2D phased arrays can be electronically focused and steered in both azimuth (x) and elevation (y) directions and hence can be used to steer beams through 3D volumetric regions, thus providing real-time volumetric imaging without the need of physically moving the transducers. 3D ultrasound imaging allows sonographers to view pathology as a volume, thereby improving the comprehension of patient anatomy. In particular, this real-time dynamic 3D ultrasound imaging also currently known as 4D imaging, offers a unique ability for cardiovascular and ocular ultrasound diagnoses. Having the real-time 3D images with ultrahigh resolution (i.e. <100 μm), the small tumors can be easily identified for detection of early stage cancer, given that blood flow in tumors is different from that in normal tissues.

Example 2D phased array ultrasound devices can be found in U.S. Pat. Nos. 5,808,967, 5,865,163, 6,419,633, 10,054,681, 10,347,818, 10,499,509, and United States Patent Publication US2020/0046320. In addition see: Zhou W, Zhang T, Ou-Yang J, Yang X, Wu D, Zhu B. PIN-PMN-PT Single Crystal 1-3 Composite-based 20 MHz Ultrasound Phased Array. Micromachines (Basel). 2020; 11(5):524. Published 2020 May 21. doi:10.3390/mi11050524.

### SUMMARY OF THE INVENTION

It is an object of the invention, in its preferred form to provide an improved form of 2D phased array ultrasound transducer.

In accordance with a first aspect of the present invention, there is provided a 2D phased array ultrasound device wherein the resulting spatial resolution is less than about 90 μm and the centre frequency is about 25 MHz.

In accordance with another aspect of the present invention, there is provided a 2D phased array ultrasound device including: a 2D array of piezo crystal elements formed from the kerfing of a single crystal; a series of conductive electrodes formed on opposed sides of the piezo crystal elements; a backing unit comprising backing filler material and a series of flexible circuit layers sandwich together to interconnect the back surface electrodes of the piezo crystal elements; and a series of front matching layers, having an acoustic impedance matching material, impedance matching the piezo crystal elements to human tissue.

Preferably, the resulting spatial resolution is less than about 90 μm and the centre frequency is about 25 MHz. In some embodiments, the thickness of the piezo crystal element is about 80 μm. In some embodiments, the backing filler material is formed from aluminium oxide and tungsten particles suspended in an epoxy resin.

In some embodiments, the transducer element includes a series of front matching layers, having an acoustic impedance matching material matching the transducer impedance to the human body. In some embodiments, the number of front matching layers is two. The thickness of the first matching layer can be about 38 μm. The thickness of the second matching layer can be about 27 μm. The front matching layers can be formed from an epoxy and filler material mix.

In accordance with a further aspect of the present invention, there is provided a 2D phased array ultrasound device including: a 2D array of piezo crystal elements formed from the kerfing of a single crystal; a series of conductive electrodes formed on opposed sides of the piezo crystal elements; and a backing unit comprising backing filler material and a series of flexible circuit layers sandwich together to interconnect the back surface electrodes of the piezo crystal elements.

Preferably, the flexible circuit layer includes a linear array of conductive interconnect elements along one edge thereof, for interconnecting with the back electrodes of the conductive electrodes.

In some embodiments, the flexible circuit layer and said filler material are formed together on a first planar substrate, before attachment to the back surface electrodes of the piezo crystal elements. In some embodiments, the backing filler material is attached to said first substrate and subsequently kerfed into a series of slots for insertion of flexible circuit layers. In some embodiments, the flexible circuit layer includes a series of conductive tabs along one proximal end thereof.

In some embodiments, the kerfed single crystal is initially filled with a structural epoxy. Preferably, an impedance matching layer is formed on top of the electrode. Preferably, a focusing layer is formed over the top of the electrode.

In accordance with another aspect of the present invention, there is provided a method of forming a 2D array of ultrasound devices, the method including the steps of: providing a planar form of piezo crystal; kerfing the planar form of piezo crystal into an array of piezo elements; filling the kerfs with a filler material; optionally thinning the back of the piezo elements; forming a first electrode on a top surface of the piezo element; forming an insulating backing material on a temporary substrate, and slotting the backing material into a series of slots; forming a series of elongated flexible printed circuit layers including piezo element connections; sandwiching of flex printed circuit elements and backing material together to form a sandwich structure; releasing the sandwich structure from the temporary substrate; forming a top conductive electrode layer on a planer end of the sandwich layer; mating the piezo elements with the electrode layer on the electrode; and dicing the filler material to separately release the electrodes and piezo elements. In some embodiments the method also includes the steps of: filling the gap between piezo elements with a non conductive material; forming at least one acoustic impedance matching sheet over the piezo elements; or forming an ultrasound focusing layer over the piezo elements.

### BRIEF DESCRIPTION OF THE DRAWINGS

Embodiments of the invention will now be described, by way of example only, with reference to the accompanying-drawings in which:

FIG. 1 illustrates a schematic cross-sectional view of the structure of 2D phased array showing wiring to each active element through flexible circuit.

FIG. 2 illustrates the dicing of the piezoelectric single crystal plate along two mutually perpendicular directions.

FIG. 3 illustrates filling the kerf with low-viscosity epoxy and polish of the surface.

FIG. 4 illustrates the grind and polish of the bottom side till achieving a desired thickness.

FIG. 5 illustrates the re-coat of the Cr/Au electrode on both surfaces of the 1-3 composite.

FIG. 6 illustrates the backing layer (insulating) which is attached on a piece of thin glass substrate for curing purposes.

FIG. 7 illustrates the dicing of the backing layer. The width of kerf is the same as the thickness of the flexible circuit. The pitch is consistent with that of the piezoelectric elements.

FIG. 8 and FIG. 9 illustrate schematic diagrams of the designed flexible printed circuit boards.

FIG. 10 and FIG. 11 illustrate the insertion of flexible circuits into the backing layer and ensure the position of each internal wire in the FPC is correct.

FIG. 12 illustrates the removal of the backing glass to reveal the internal wires in the FPC are visible on the surface of the backing layer.

FIG. 13 illustrates a corresponding photo for the arrangement of FIG. 12.

FIG. 14 illustrates attaching the piezoelectric 1-3 composite onto the backing layer using conductive adhesive. FIG. 15 illustrates a corresponding photo.

FIG. 16 illustrates cutting of the E-Solder layer in order to separate each individual element electrically and acoustically. FIG. 17 illustrates a corresponding photo.

FIG. 18 illustrates re-filling of the kerfs with epoxy

FIG. 19 illustrates attaching two matching layers on the top of the 1-3 composite.

FIG. 20 illustrates attaching focusing lens on the matching layers.

FIG. 21 and FIG. 22 illustrate the design of the integrating printed circuit board.

FIG. 23 and FIG. 24 illustrate photographs of the connection between the FPC and PCB.

FIG. 25 illustrates a schematic diagram of the designed bridging PCB boards;

FIG. 26 illustrates a photograph of the case of the fabricated 2D phased array ultrasonic transducer with custom-made housing.

FIG. 27 illustrates the transducer in an assembled form.

FIG. 28 illustrates the resultant electrical impedance and the phase angle spectra of the 2D array transducer prototype.

FIG. 29 illustrates the pulse-echo response and frequency spectrum of the 2D phased array transducer.

FIG. 30 illustrates the wire phantom generated using the signals obtained from all the array elements working simultaneously. The image has been inverted for reproducibility.

FIG. 31 illustrates a cross-sectional view (XOZ) of the wire phantom under phase-focus scanning mode. The image has been inverted for reproducibility.

FIG. 32 illustrates a front view (YOZ) of the wire phantom under phase-focus scanning mode. The image has been inverted for reproducibility.

FIG. 33 illustrate 3D images of the wire phantom captured from a 4D imaging view. The image has been inverted for reproducibility.

FIG. 34 illustrates a cross-section view of the phantom under a plane-wave scanning mode using 16 elements in one line of the 2D array transducer. The image has been inverted for reproducibility.

FIG. 35 illustrates an estimation of the axial resolution through a MATLAB program.

FIG. 36 illustrates an estimation of the lateral resolution through a MATLAB program.

### DETAILED DESCRIPTION

The preferred embodiments provide a 256-element two-dimensional (2D) phased array ultrasonic transducer capable of high resolution imaging based on piezoelectric single crystals for real-time three-dimensional (3D) medical imaging, in particular for ocular and cardiovascular disease diagnosis.

The fabrication process of the transducer primarily involves a dice-and-fill of piezoelectric single crystal into 1-3 composite that is connected to the designated channels of an imaging system through wiring the individual piezoelectric elements with the flexible printed circuits. The developed transducer, consisting of 256 array elements, exhibits a high central frequency of 25 MHz, a relatively broad bandwidth of 48% and a low insertion loss of −36 dB, collectively leading to a high spatial resolution i.e. lateral resolution=67 μm and axial resolution=90 μm, obtained from a 3D imaging test, which are sufficient to image the 3D fine structures of the fluid channels in human eyes and hearts. In this sense, the embodiments can have significant clinical impact on the understanding of pathogenesis and diagnosis of many ocular and cardiovascular diseases relating to the fine structures of organs, such as glaucoma, pars planitis, coronary heart disease, vascular dementia and strokes etc.

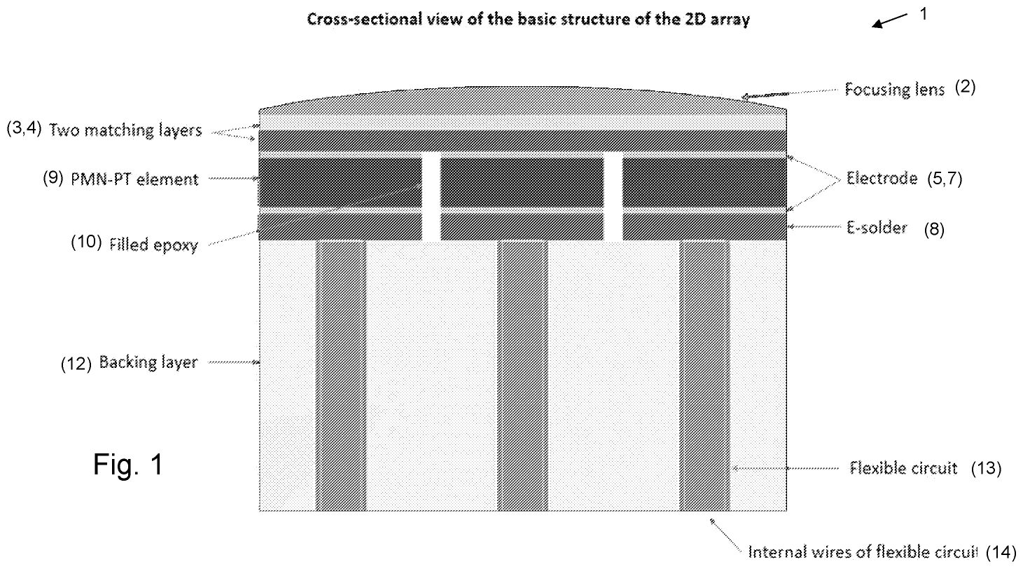

Turning initially to FIG. 1, where there is illustrated a schematic sectional view through part of the 2D array of phased array elements 1.

The array includes a first focusing lens 2, to which is attach to two acoustic impedance matching layers 3,4, which are designed to impedance match the acoustic signal to the human skin. Next, the piezoelectric material 9 is provided, sandwiched between electrodes 5, 7. E-Solder 8 connects one of the electrodes to the internal wires 14 of a flexible circuit board 13. The regions between the piezo material are filled with an epoxy 10. A backing layer 12 provides not only structural support but also damping to efficiently shorten the duration of resonant vibration, thus improving the axial resolution of the transducer.

The embodiments include a prototype of a high frequency 2D phased array ultrasonic transducer and an interface to connect each individual piezoelectric array elements in a bulk process. Accordingly, the prototype provides a high performance 2D phased array transducer prototype which is capable of real-time imaging of 3D fine structures at a scale of several tens of microns.

### Fabrication Process

The 2D array ultrasonic transducer arrays are fabricated from a block of piezoelectric single crystal within which individual elements are defined by making a number of cuts through the block of the crystal using a high precision dicing saw.

In order to observe fine structure of human body, the centre frequency of the transducer needs to be at least about 20 MHz, i.e the thickness of the active layer needs to be about 80 μm. A 2D array with 256 elements (16×16) was used as the active layer for a prototype, although other arrangements could be utilised. The detailed fabrication process is described as follows.

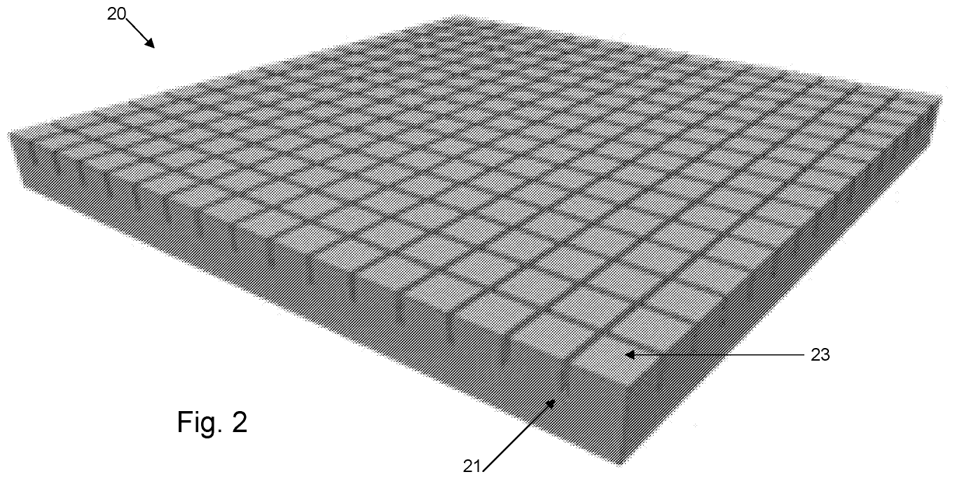

1. Poling of the piezoelectric layer: Turning initially to FIG. 2, a single crystal of piezo crystal material 20 is shown. The size of the starting piezoelectric single crystal 20 is 7×7×3 mm (w×l×t). The crystal plate is electrode coated with chrome and gold with a thickness of ˜700 nm on both surfaces. The longitudinal length vibration mode of the crystal will be preferably used in the transducer because the corresponding piezoelectric coefficient (d₃₃) is higher than those of other modes. Poling of the crystal is conducted under a de electric field in the bath of silicon oil at room temperature.

2. Cutting of the arrays: The 2D array is then scored or cut 21. The cutting of the 2D arrays can be conducted using a precision dicing saw. Cuts are made along both azimuthal (x) and elevation (y) direction with the pitch size of 383 μm including the kerf of ˜13 μm wide generating 256 (16×16) elements 23. The plate is partially sliced rather than cutting through to ensure that the sliced elements are rigidly held together by a solid layer at all stages of fabrication.

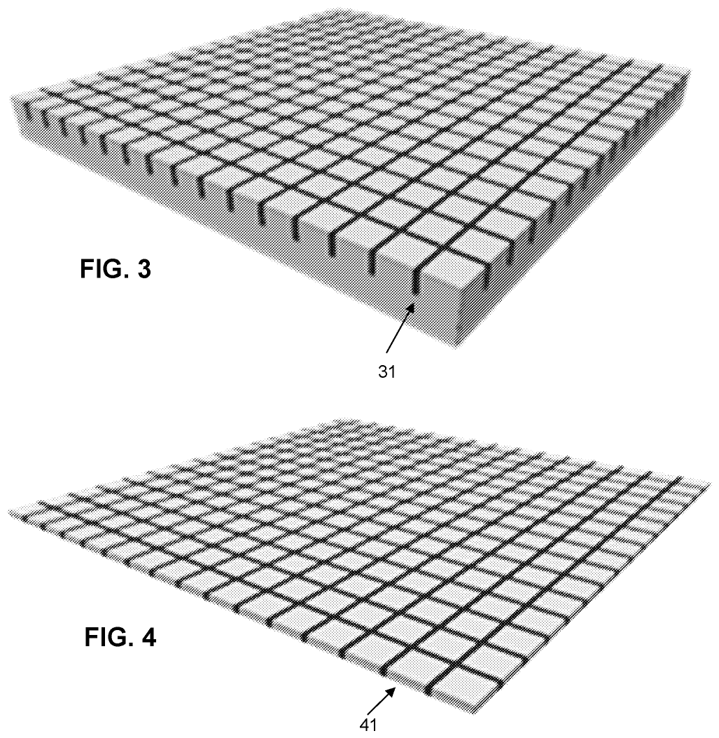

3. Filling kerfs: Next, as illustrated in FIG. 3, the kerfs, or cuts are filled with low-viscosity insulating epoxy e.g. 31. After curing, the surface is polished to remove the excess of epoxy.

4. Grinding of the active layer: As illustrated in FIG. 4, the piezoelectric crystal is further ground 41 to remove the uncut part till a desired thickness. Thus, the fabrication of a 1-3 composite is completed.

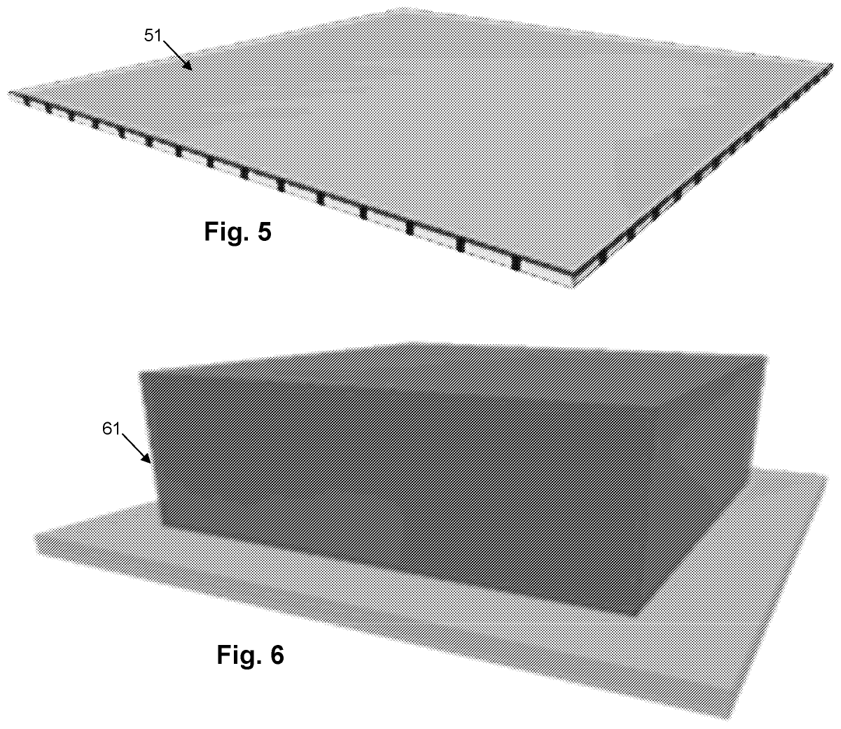

5. Electrode sputtering: Next, as illustrated in FIG. 5, the both sides of the 1-3 composite have been ground in steps 3 and 4, a Cr/Au electrode is re-coated on both surfaces 50 for wiring purpose subsequently.

6. Fabrication of backing layer: Next, as illustrated in FIG. 6, the insulating backing layer 61 is made from Al₂O₃ and tungsten particles co-loaded epoxy. The ratios of the ingredients are tuned to achieve the desired damping effect, i.e. attenuation of the acoustic waves emitting backwards.

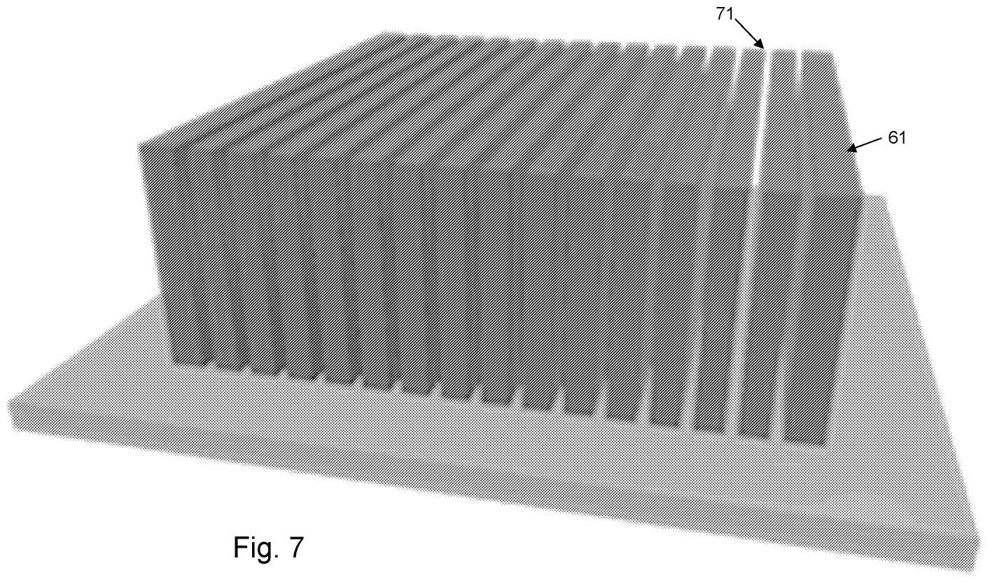

7. Cutting of the backing layer: As illustrated in FIG. 7, the backing layer is ground till the thickness is about 2 mm and then diced through 71 along one direction only with the same pitch size as that of the piezoelectric 1-3 composite. The width of the cutting kerf 71 is determined based on the thickness of the intended inserted flexible printed circuit boards (FPC) (see below).

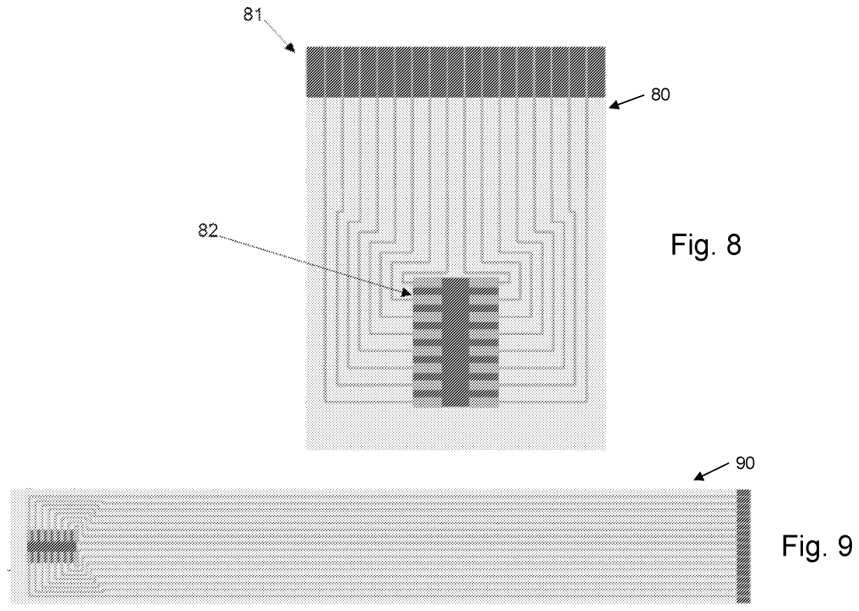

8. Design of flexible printed circuit (FPC) board: As shown in FIG. 8 and FIG. 9, 16 FPC boards 80, 90, each of which contains 16 internal electrical wires, are inserted into the kerfs 71 of the backing layer of FIG. 7, making a 16×16 wire arrays with the same pitch as that of the piezoelectric elements. One end of the FPC board (end 81 in FIG. 8), where the internal wires are exposed, connects to the back electrode of the piezoelectric elements for transmitting the electrical pulse signals. The FPC boards should be as small as possible to minimise the negative impact of the reduced volume of backing layer. It is ideal to be less than 50 μm by considering the current industrial level and the cost. This is relatively thin compared to the 370 μm-wide elements.

On the other side of the FPC, the 16 printed wires end at 16 respective rectangular connector pads (pads 82 in FIG. 8), allowing further connection of the printed wires to paired connectors on printed circuit boards (PCB). The adoption of FPC board significantly simplified the wiring process of 2D array transducers.

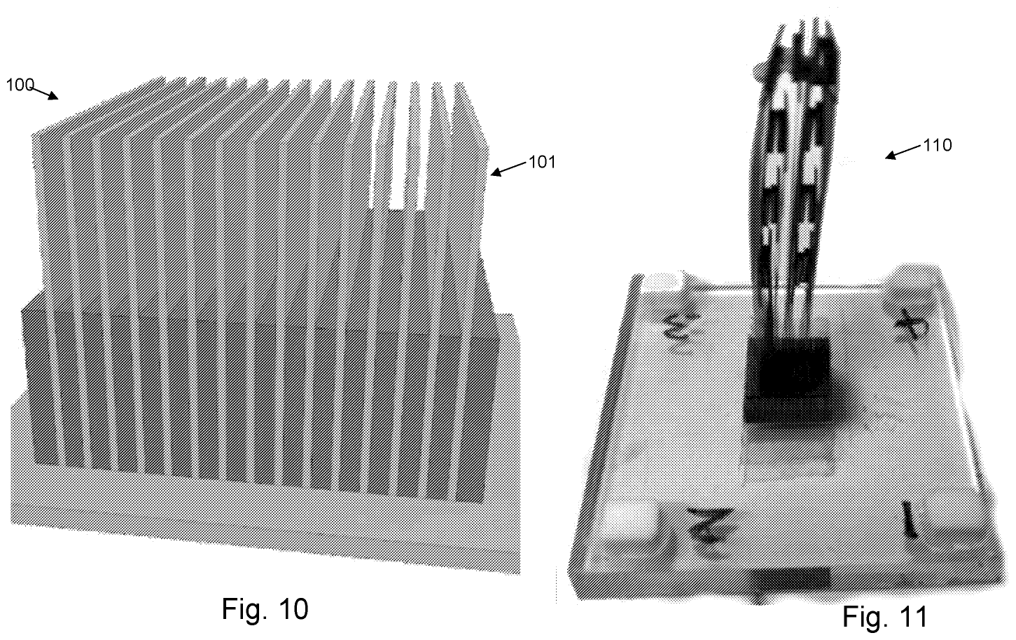

9. Insertion of FPC boards into backing layer: Next as illustrated 100 in FIG. 10, the FPC boards e.g. 101 are then inserted into the kerfs in the backing layer. It is important that all the FPC are inserted to the very end of the backing layer touching the glass that holds the backing layer. After insertion, all the kerfs will be filled with insulating epoxy to fix the FPC in the backing layer. FIG. 11 illustrates a photo 110 of the formation of the prototype.

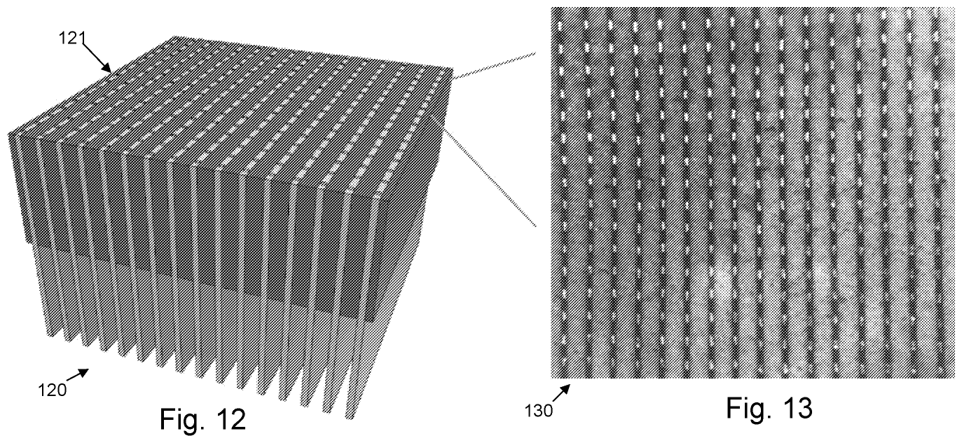

10. Removal of backing layer from glass substrate: FIG. 12 illustrates 120 the backing layer is taken off from the glass substrate and slightly ground to ensure all the internal wires are visible on the surface 121 of the backing layer. Thus, each wire in FPC boards is in the correct position and will be connected with one piezoelectric array element only. FIG. 13 illustrates a corresponding photo 130 of a prototype for FIG. 12.

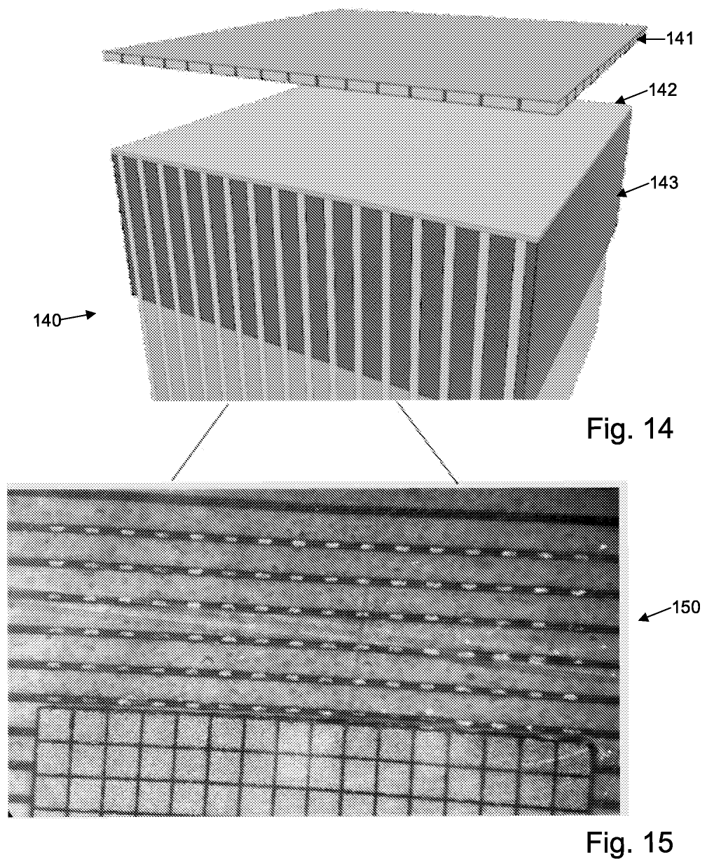

11. Attachment of 1-3 composite on backing layer: Next, as illustrated 140 in FIG. 14, the 1-3 composite layer 141 is glued on the backing layer 143 by conductive E-solder 142, which is a mixture of silver particles and epoxy with curing agent. The centre of each element is aligned to the wires. This step guarantees the electrical contact from the bottom electrode of the elements to the wires. FIG. 15 illustrates a photograph 150 of the prototype arrangement.

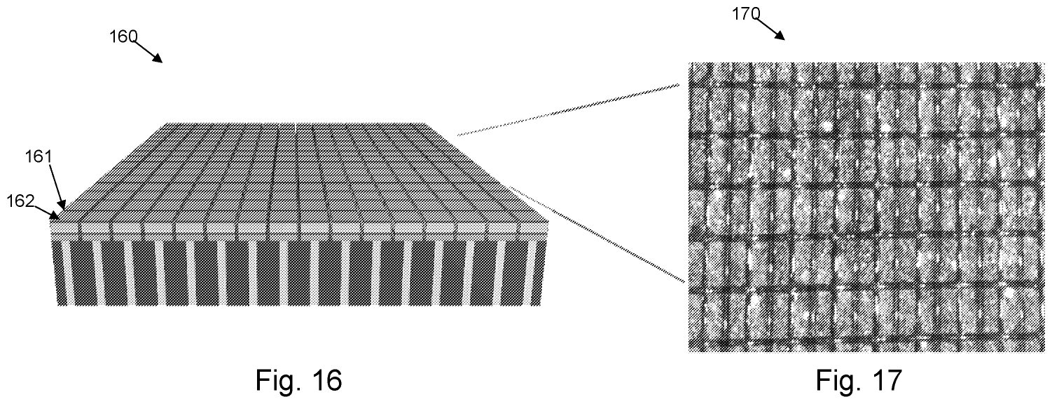

12. Electrical separation of array elements: Turning now to FIG. 16, after curing, the E-solder layer needs to be cut e.g. 161 for electrical and acoustic separation of array elements e.g. 162. To this end, the cutting can be carried out from the epoxy filler in the kerfs of the piezoelectric 1-3 composite with a thinner dicing blade (˜10 μm thick). The epoxy filler along with the E-solder layer can be cut through till the blade edge reaches the surface of the backing layer. This step is designed to separate the conductive E-solder layer into small individual electrodes such that each single array element will be electrically and acoustically separated. FIG. 17 illustrates a corresponding photo 170.

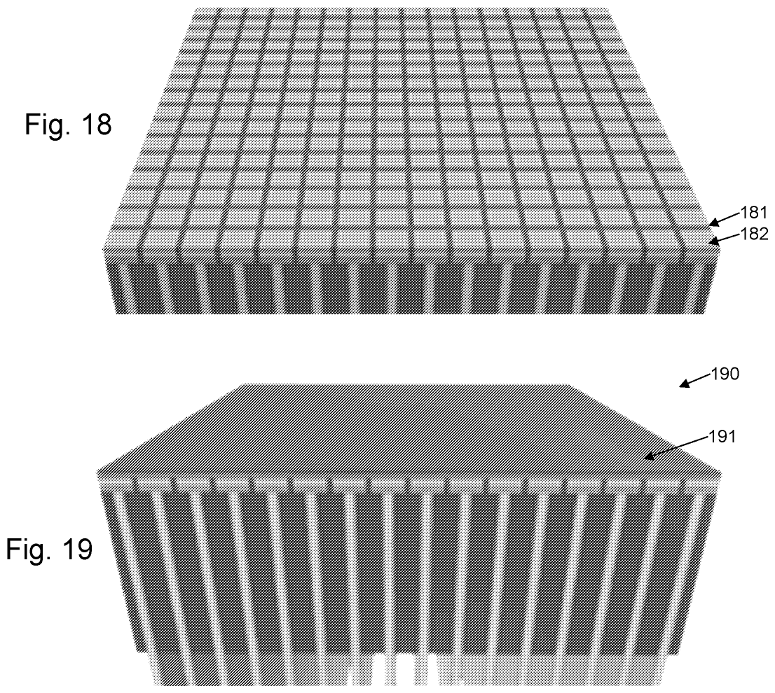

13. Kerfs re-filling: As illustrated in FIG. 18, after cutting the thin E-solder layer in Step 12, the kerfs in the 1-3 composite are re-filled with insulating epoxy e.g. 181. The top surface e.g. 182 of the 1-3 composite can be repolished to remove excess of epoxy and re-coated with Cr/Au electrode acting as the common ground for all active elements.

14. Development of front matching layers: Next, as illustrated 190 in FIG. 19, the matching layers 191 are used to increase the transmittance of ultrasound from the transducer to human body, thus improving the bandwidth and resolution of the transducer. A bi-layered structure is adopted as the matching layers in this prototype. Thickness and acoustic impedance of the two matching layers can be determined through the calculation results based on a Krimholtz-Leedom-Matthaef (KLM) model. Both matching layers 191 are made from epoxy based materials with different fillers. The first matching layer can be casted and manually ground to the desired thickness. The second matching layer can be painted on the first matching layer. After the curing, the second matching layer is ground into appropriate thickness.

In one embodiment, the active later thickness was 75 μm, the first matching layer thickness was 38 μm, the second matching layer thickness was 27 μm and the backing layer thickness was 1.69 mm.

15. Attaching matching layers: The double matching layers are attached to the top surface of the 1-3 composite with insulating epoxy and then held to be pressed in a custom made mould for an extended time of period in order to dry the adhesive layer and control its thickness to be several microns.

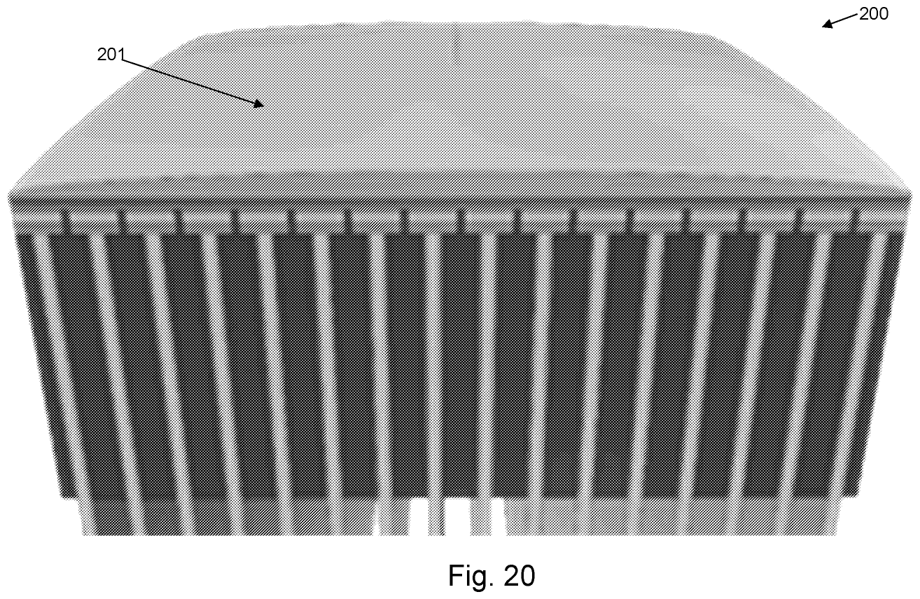

16. Attaching focusing lens: As illustrated in FIG. 20, an acoustic-impedance-matching focusing lens layer is mounted onto the transducer surface to allow a better focus of the beam in order to obtain a better lateral resolution.

### Integrated Circuit Board Design

17. The design of the integrating printed circuit board: The 16 previously inserted FPC boards containing 256 wires that are connected to two integrated printed circuit boards (PCB), i.e. 128 wires per PCB.

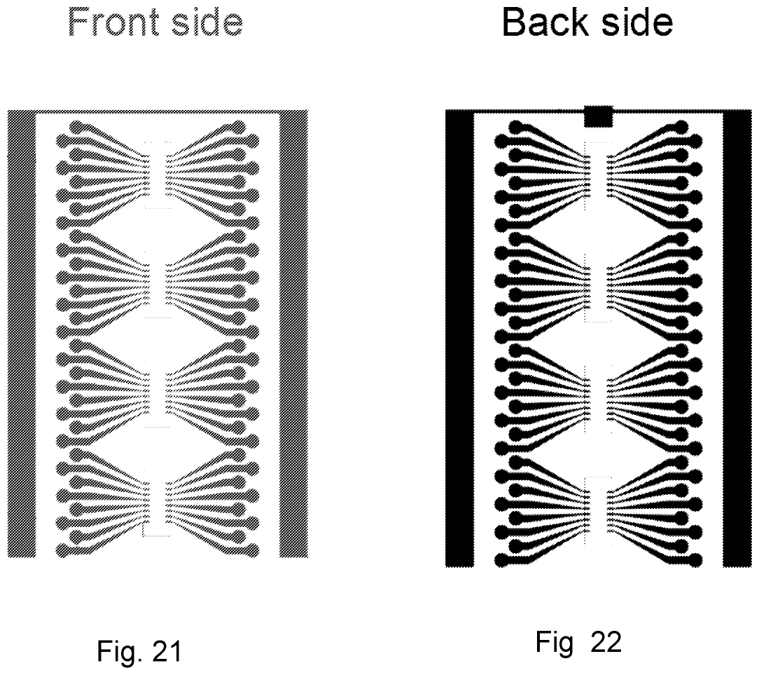

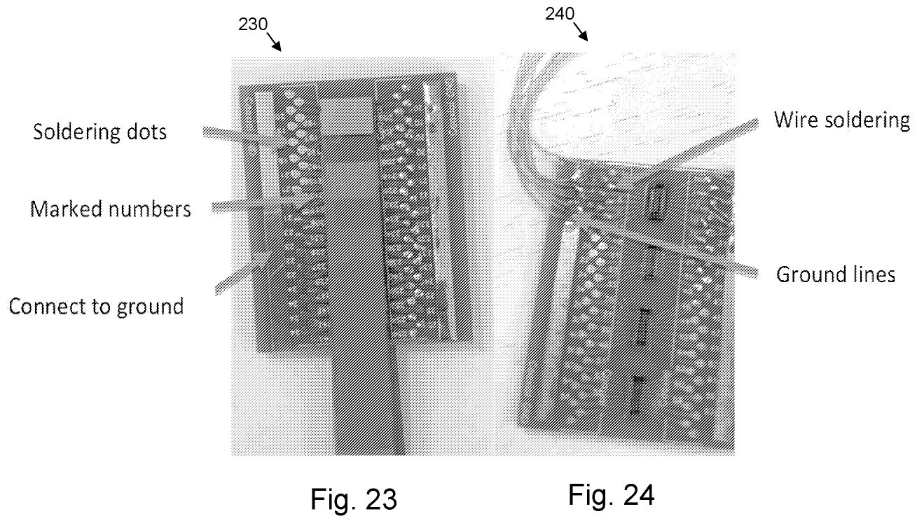

As shown in FIG. 21 and FIG. 22, there are 4 connector sockets, which are paired to the connectors on the FPC boards, in the middle of both surfaces of the PCBs. The connectors on FPC boards of different length are plugged into the sockets on respective PCB. FIG. 23 and FIG. 24 illustrate photographs 230, 231 of the resultant PCB connections.

These two PCBs need to be further connected to the imaging system. To this end, each of the pins of the connectors on PCBs are connected to individual soldering dots through wire bonding technique, as marked in FIG. 23 and FIG. 24. External wires are soldered to these dots and the ground lines are connected on the two bar-shaped soldering area as shown in FIG. 23 and FIG. 24. In order to effectively control the excitation of individual piezoelectric element and distinguish the signals from each element, all the soldering dots are numbered.

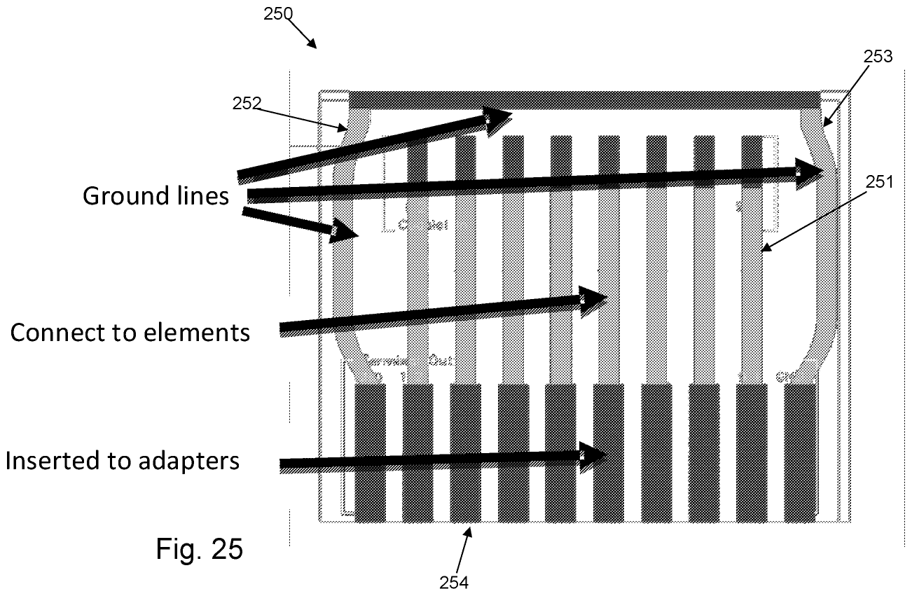

18. Connecting to the imaging system terminal: As illustrated in FIG. 25, the wires from the numbered soldering dots on the two PCBs are soldered to 16 bridging PCBs, (FIG. 25) which connect to the imaging terminal. There are 8 equally spaced straight bar-pads e.g. 251 on the bridging PCB connecting to the array elements on each end. Thus, one bridging PCB connects 16 elements and 16 such bridging PCBs are needed for connecting all the 256 elements. The two slightly curved pads e.g. 253 near the edges of the bridging PCB are grounded. The 10 bars at the bottom e.g. 254 will be plugged into the adapters to the imaging terminals.

19. Packaging: Since there are various electromagnetic noises in the environment which may influence the transducer signals, an external shield made from copper foil is used to cover all wires and cables. The shielding foil is also grounded. Finally, the 2D phased arrays are assembled into a custom-made housing, a prototype 2D phased array ultrasonic transducer is completed.

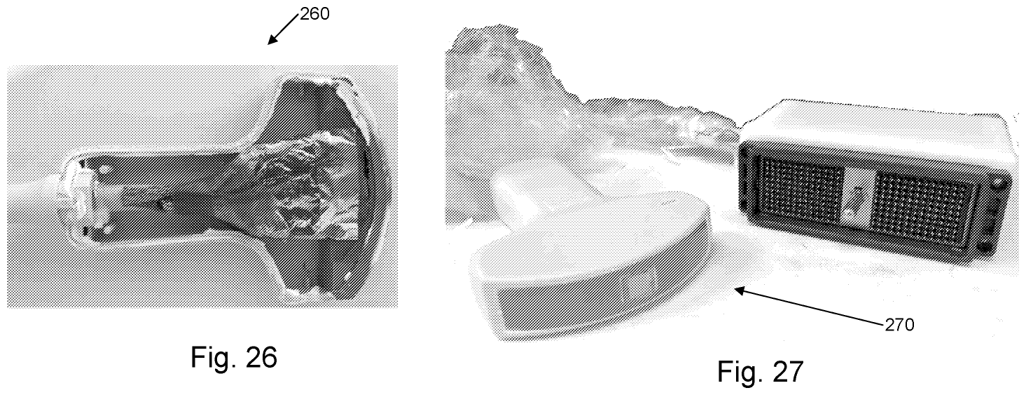

FIG. 26 illustrates a photograph of the case of the prototype fabricated 2D phased array ultrasonic transducer with custom-made housing. FIG. 27 illustrates the prototype transducer 270 in an assembled form.

The electrical impedance resonance spectrum, pulse-echo response and bandwidth of the prototype array element were experimentally obtained and measured. The 2D array transducer was immersed in water during these measurements. Water is used as the loading medium due to its similar acoustic impedance (1.5MRayls) to those of biological tissues (1.5-2.0MRayls).

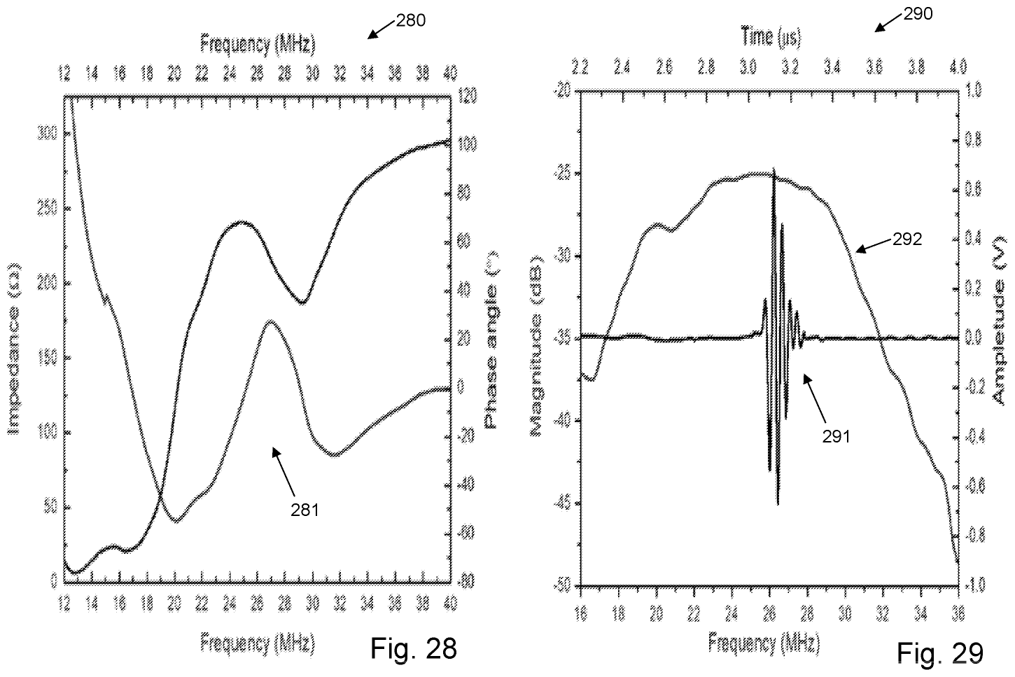

FIG. 28 shows the measured impedance and phase spectra 280 of the fabricated 2D array transducer. The central frequency 281 was determined to be ˜25 MHz. Based on the resonance frequency fᵣ and anti-resonance frequency fₐ obtained from the spectra, the electromechanical coupling coefficient kₜ is calculated to be 0.67, which is relatively high, allowing an efficient conversion between electrical and mechanical energy. The measured electrical impedance at resonance frequency is 40.2 Ω, which is very close to the desired impedance of 50 Ω. This enables an effective electrical energy transmission between the operation system and the transducer. The phase angle change is ˜40°, which can lead to sufficient sensitivity so that magnitude of electrical signals travelling in/out of the transducer is maximized.

FIG. 29 illustrates 290 the pulse-echo response 291 and the corresponding frequency spectrum 292 obtained by Fast Fourier Transform (FFT). It can be seen that the echo exhibits a peak-to-peak value of 1.4 V with a pulse length of 128 ns. The amplitude of the echo signal is sufficiently strong to provide desired sensitivity and image quality. The −6 dB bandwidth is determined to be 48% within the frequency range from 18.5 MHz to 30.5 MHz. The center frequency is found to be 25 MHz, and the insertion loss (IL) of the transducer is measured to be −36 dB. These performance are satisfied for commercial applications.

An imaging test on the 2D array transducer was performed using a wire phantom consisting of 5 tungsten filaments fixed on a stainless steel base. The diameter of the tungsten filaments in this phantom is 12.5 μm. The tungsten filaments are equally spaced with 0.25 mm and 0.5 mm gap in vertical and horizontal directions, respectively. Both 2D array transducer and the phantom were immersed into water during imaging test. The distance between the transducer and the phantom is 7 mm.

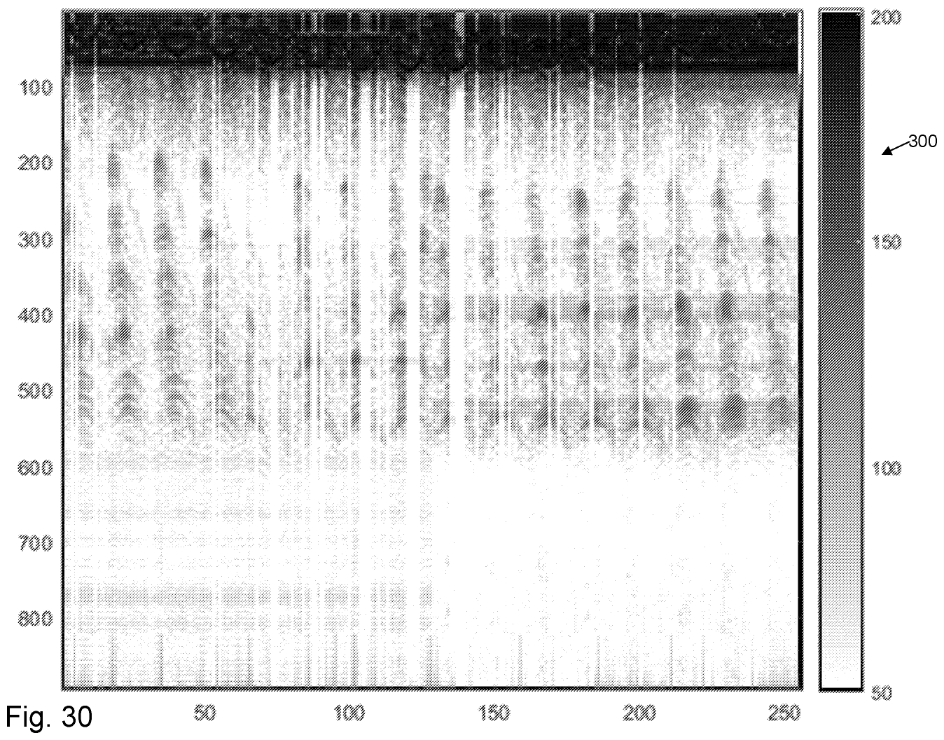

The uniformity and the percentage of functioning elements are measured first. FIG. 30 shows the image produced 300 using the signals from all the 256 array element by scanning the wire phantom at the same time without any treatment by the imaging system. The image has been inverted for reproducibility. Although there are several weak-signal points, up to 90% of the elements are able to work properly.

Crosstalk between elements, which is the signal emitted from one element received by other elements particularly the adjacent element when it is reflected back, should be as small as possible. The crosstalk of the nearest elements is found to be −26 dB.

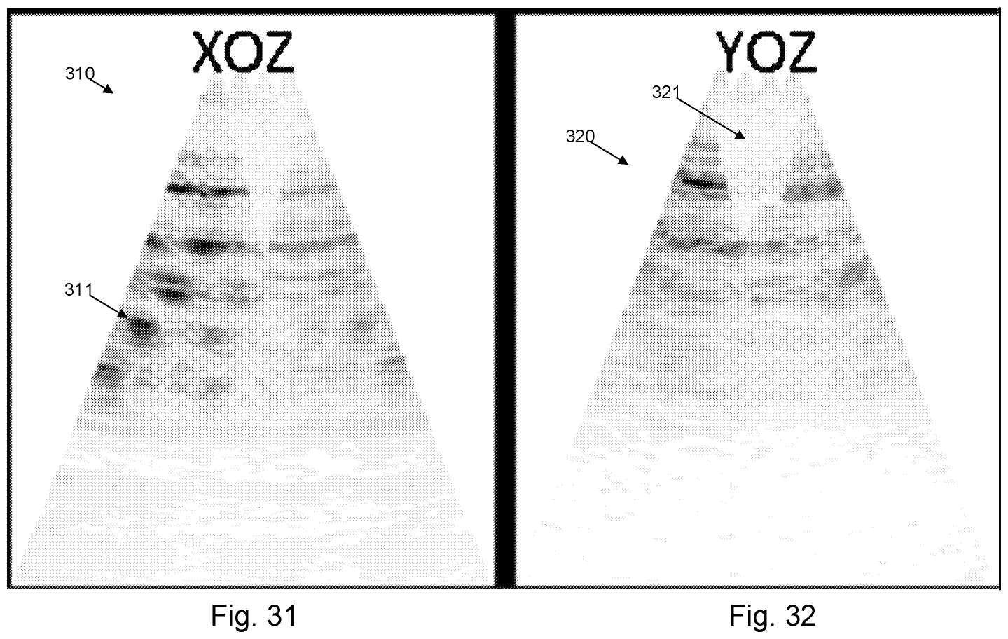

FIG. 31 and FIG. 32 present the 2D slice view of the tungsten wire phantom in XOZ (310) and YOZ plane (320) generated by the fabricated transducer. The images have been inverted for reproducibility. The image of XOZ plane is a cross section view that perpendicular to the tungsten filaments manifested in several bright dots. There are 4 obvious bright dots representing the last 4 wires, which can be seen e.g. 311 in FIG. 31 ranged in a certain scan line on the left side of the XOZ image. However, the dot representing the first tungsten filament (at the furthest end from the transducer) is less conspicuous.

There is also a missing line at the furthest end in the front view image (YOZ), which should render 5 recognisable lines with equal interval. In addition to natural attenuation of the ultrasound with the increase of emission distance, the low sampling rate of the imaging system is responsible for the weak/missing signal 321 in the image shown in FIG. 32.

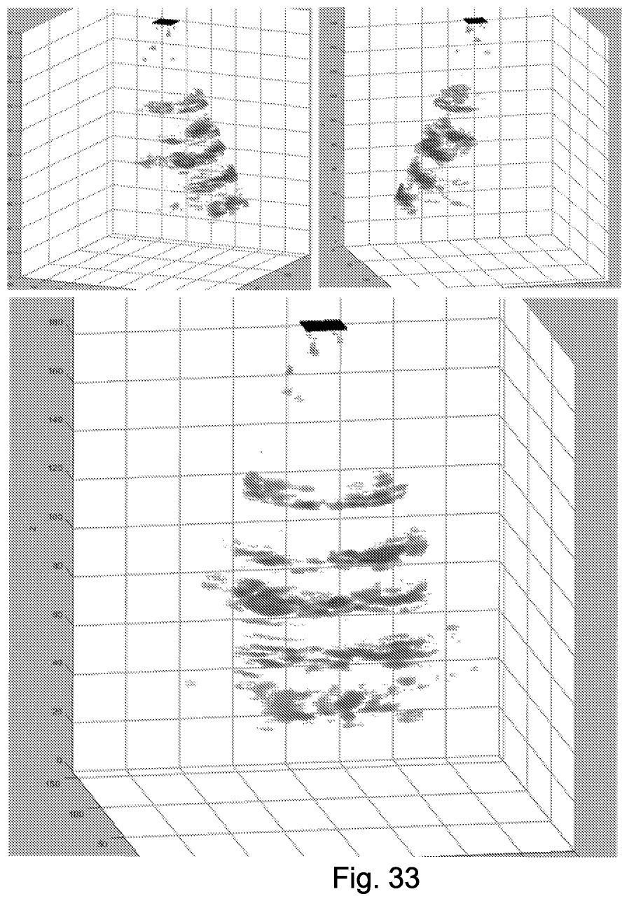

Several 3D images of the wire phantom captured from a 4D image are shown in FIG. 33. The 4D image is a real-time 3D image over a certain period of time. The displayed images are captured at different time showing the wire phantom at different angles. All the five tungsten filaments can be observed in these 3D images.

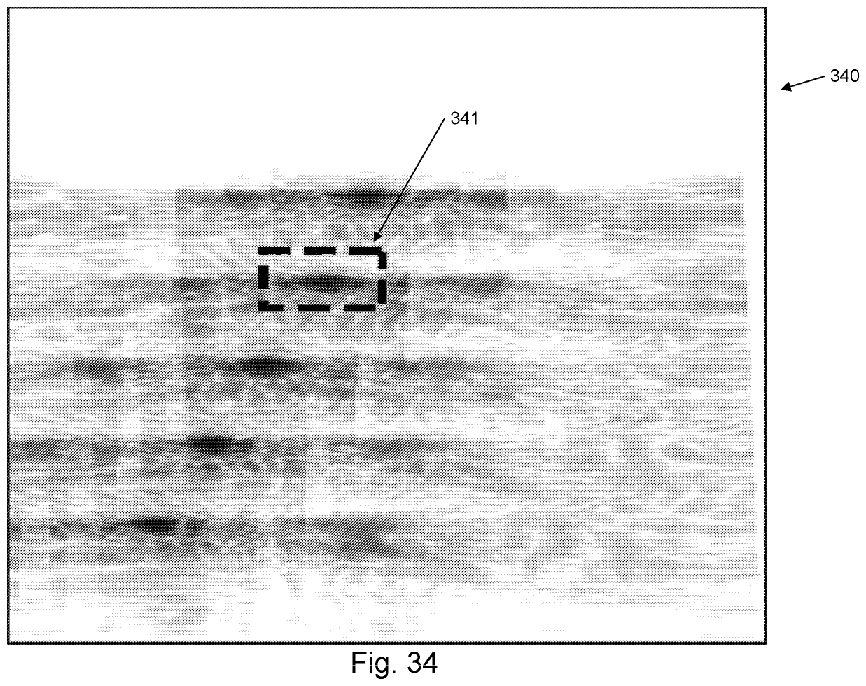

In order to obtain a clearer image with the relatively low sampling rate of the existing imaging system, the wire phantom was re-imaged using only 16 elements in one line in the 2D array transducer. The resulted cross-sectional view (2D) of the wire phantom is shown 340 in FIG. 34. All the five tungsten wires are well resolved and clearly observed in the cross-section view e.g. 341, implying a high spatial resolution of the transducer.

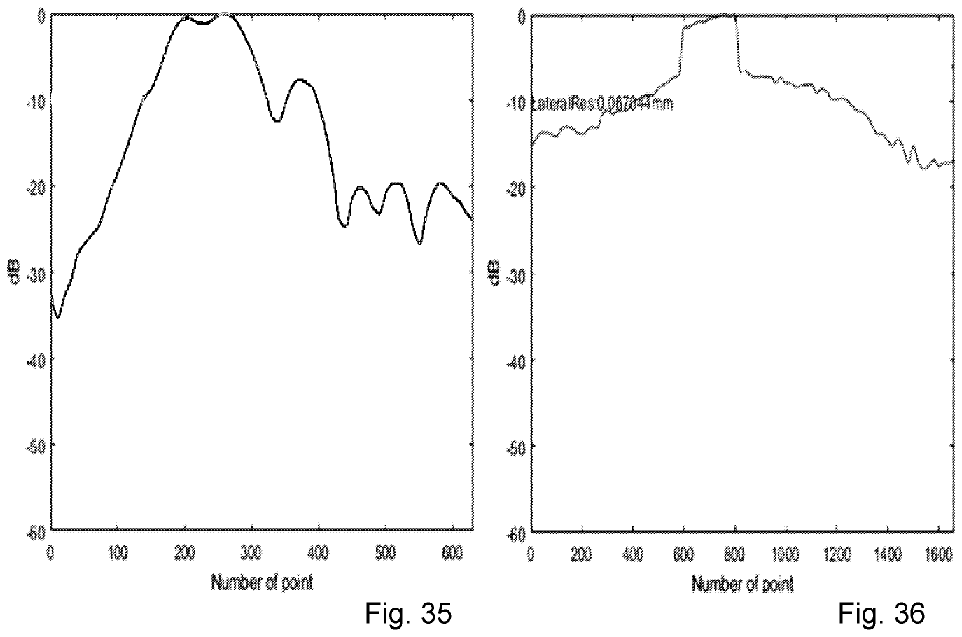

The resolution was estimated using the highlighted dot 341 in FIG. 34. As shown in FIG. 35, the axial resolution is found to be 90 μm at −6 dB, while the lateral resolution (FIG. 36) was 67 μm.

The fabricated 2D phased array ultrasonic transducer exhibits extremely high spatial resolution without any obvious side lobes near the main signal as evidenced by the imaging test. The remarkable resolution can be attributed to the high central frequency (˜25 MHz) that results in shorter wavelength and shorter pulse length in conjunction with the effective damping arising from the backing layer. In summary, the fabricated 2D array transducer successfully generates clear wire phantom images and a promising real-time 3D view even in case that frequencies of the imaging system and transducer are not perfectly matched. These results provide a well-founded hope that the 2D phased array transducer in this invention is capable of producing very high quality 3D/4D images of fine structures at scales of several tens microns, provided the sampling rate of the imaging system matches the high frequency of the transducer.

The embodiments provide a 3D imaging technology which provides for accurate, efficient, and real-time diagnostic medical sonography. The 3D imaging generated by 2D phased arrays exhibits many advantages, including improved axial resolution, high frame rates, lower side lobes, less noise in the near field and outstanding quality of images, which are highly demanded for enhanced echocardiography workflow and optimal volumetric imaging in cardiovascular and ocular diagnostic applications.

The prototype of 2D phased array ultrasonic transducer not only possesses high resolution in volumetric imaging, but offers additional benefits, such as compact size and facile wiring technique, making it viable for producing high quality real-time 3D imaging of fine structures, and provides high performance, low fabrication difficulties, and portability of future 3D ultrasonic imaging technologies.

The prototype of high-frequency 2D phased array ultrasonic transducer represents a promising technology in real-time diagnostic medical sonography through efficiently generating high-resolution volumetric imaging. This technology is particularly suited for the diagnosis of ocular and cardiovascular diseases, which often require evidence from 3D imaging of fine structures. The developed 2D array transducer can also be used for early detection of cancer and tumours thanks to its high spatial resolution. The unique interface employing flexible printed circuit greatly reduces the complexity of manufacturing of 2D phase arrays with small pitch and avoids the damages of piezoelectric elements during the conventional wire-soldering process.

### Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

We claim:

1. A 2D phased array ultrasound transducer device including:
a 2D array of piezo crystal elements formed from the kerfing of a single crystal;
a series of conductive electrodes formed on opposed sides of the piezo crystal elements;
a backing unit comprising backing filler material and a series of flexible circuit layers sandwich together to interconnect the back surface electrodes of the piezo crystal elements; and
a series of front matching layers, having an acoustic impedance matching material, impedance matching the piezo crystal elements to human tissue.

2. A 2D phased array ultrasound transducer device as claimed in claim 1 wherein the resulting spatial resolution is less than 90 μm and the centre frequency is about 25 Mhz.

3. A 2D phased array ultrasound device as claimed in any previous claim wherein the thickness of the piezo crystal element is about 80 μm.

4. A 2D phased array ultrasound device as claimed in any previous claim wherein the backing filler material is formed from aluminium oxide and tungsten particles suspended in an epoxy resin.

5. A 2D phased array ultrasound device as claimed in any previous claim wherein the transducer element includes a series of front matching layers, having an acoustic impedance matching material matching the transducer impedance to the human body.

6. A 2D phased array ultrasound device as claimed in claim 5 wherein the number of front matching layers is two.

7. A 2D phased array ultrasound device as claimed in claim 6 wherein the thickness of the first matching layer is about 38 μm.

8. A 2D phased array ultrasound device as claimed in claim 6 wherein the thickness of the second matching layer is about 27 μm.

9. A 2D phased array ultrasound device as claimed in any previous claim wherein the front matching layers are formed from an epoxy and filler material mix.

10. A 2D phased array ultrasound device producing ultrahigh spatial resolutions of less than about 90 μm and a high centre frequency of about 25 Mhz.

11. A 2D phased array ultrasound device as claimed in claim 1 further wherein said flexible circuit layer includes a linear array of conductive interconnect elements along one edge thereof, for interconnecting with the back electrodes of the conductive electrodes.

12. A device as claimed in claim 11 wherein said flexible circuit layer and said filler material are formed together on a first planar substrate, before attachment to the back surface electrodes of the piezo crystal elements.

13. A device as claimed in claim 11 wherein the flexible circuit layer includes a series of conductive tabs along one proximal end thereof.

14. A 2D phased array ultrasound device including:
a 2D array of piezo crystal elements formed from the kerfing of a single crystal;
a series of conductive electrodes formed on opposed sides of the piezo crystal elements;
a backing unit comprising backing filler material; and
a series of flexible circuit layers sandwich together to interconnect the back surface electrodes of the piezo crystal elements.

15. A device as claimed in claim 14, wherein the flexible circuit layer includes a linear array of conductive interconnect elements along one edge thereof, for interconnecting with the back electrodes of the conductive electrodes.

16. A device as claimed in claim X1, wherein the flexible circuit layer and said filler material are formed together on a first planar substrate, before attachment to the back surface electrodes of the piezo crystal elements.

17. A device as claimed in claim X1, wherein the backing filler material is attached to said first substrate and subsequently kerfed into a series of slots for insertion of flexible circuit layers.

18. A device as claimed in claim X1, wherein the kerfed single crystal is initially filled with a structural epoxy.

19. A device as claimed in claim X1, further comprising an impedance matching layer formed on top of the electrode.

20. A device as claimed in claim X1, wherein a focusing layer is formed over the top of the electrode.

21. A method of forming a 2D array of ultrasound devices, the method including the steps of:
providing a planar form of piezo crystal;
kerfing the planar form of piezo crystal into an array of piezo elements;
filling the kerfs with a filler material;
optionally thinning the back of the piezo elements;
forming a first electrode on a top surface of the piezo element;
forming an insulating backing material on a temporary substrate, and slotting the backing material into a series of slots;
forming a series of elongated flexible printed circuit layers including piezo element connections;
sandwiching of flex printed circuit elements and backing material together to form a sandwich structure;
releasing the sandwich structure from the temporary substrate;
forming a top conductive electrode layer on a planer end of the sandwich layer;
mating the piezo elements with the electrode layer on the electrode; and
dicing the filler material to separately release the electrodes and piezo elements.

22. A method as claimed in claim x5, further comprising:
filling the gap between piezo elements with a non conductive material;
forming at least one acoustic impedance matching sheet over the piezo elements or forming an ultrasound focusing layer over the piezo elements.
